(19) United States
(12) Patent Application Publication
(10) Pub. No.: US 2026/0185030 A1
(43) Pub. Date: Jul. 2, 2026

(54) LIQUID SAMPLING DEVICE

(71) Applicant: Agency for Science, Technology and Research, Singapore (SG)

(72) Inventors: Ying Ying WU, Singapore (SG); Ahmad Amirul Bin ABDUL RAHIM, Singapore (SG)

(21) Appl. No.: 19/128,098

(22) PCT Filed: Nov. 7, 2023
(86) PCT No.: PCT/SG2023/050734

§ 371 Date: May 7, 2025

(30) Foreign Application Priority Data
Nov. 7, 2022 (SG) 10202251655S

(51) Int. Cl. C12M 1/26; C12M 1/00; C12M 1/12; C12M 1/36

(52) U.S. Cl. CPC C12M 33/00; C12M 23/40; C12M 37/00; C12M 41/48

(57) ABSTRACT

A device for aseptic small volume sampling of liquid, comprising a flow control system and a tubing manifold. The tubing manifold comprises an air inlet, a sample inlet, an air outlet, and a sample outlet, each of which is fluidly coupled to the tubing manifold, and operatively coupled to the flow control system. The flow control system (FCS) is operable in a forward flow configuration to pump a predetermined volume of fluid from the sample inlet to the sample outlet. The flow control system is operable, after pumping the predetermined volume of fluid, in a reverse flow configuration to return fluid remaining in the tubing manifold back to the sample inlet. The forward flow and reverse flow configurations substantially prevent air bubble formation in fluid exiting the sample outlet or the sample inlet.

### TECHNICAL FIELD

The present invention relates, in general terms, to a device for aseptic small volume sampling of liquid. In particular, the present invention relates to, but is not limited to, aseptic small volume sampling where bulk fluid can be returned to a bulk fluid receptacle while maintaining sterility on the bulk fluid.

### BACKGROUND

Current cell sampling methods require manual and open manipulation of cell culture. This can introduce contaminants. Yet, sampling is a key step that is frequently performed in bioprocessing and cell manufacturing as cells are monitored through off-line assays. Non-destructive on-line monitoring is not the standard practice for these industries.

A traditional solution for cell sampling is to use an aseptic technique performed in a BioSafety Cabinet (BSC). Using aseptic techniques, the cell culture vessel is opened in a BSC so that a micropipette or serological pipette can be used to remove the sample. However, open manipulation presents opportunities for contamination and is contingent on the aseptic technique of a trained operator; manual processes require the full attention of a trained operator; use of a BSC requires overhead costs and time; access to cell culture is required and this is not compatible with closed automated systems; and moving the culture between the incubator and BSC disrupts the optimal conditions for the culture.

Another approach involves sampling through needleless swabbable valves. Needleless connectors are swabbed with alcohol wipes and connected to create a fluid path for the extraction of sample volume. However, the tubing retains a dead volume of sample and this volume has to be flushed out before the next sample is taken. This wastes cell sample as the cell culture remaining in the tubing cannot be aseptically returned to the cell culture; previous samples left in the tube can contribute to inaccuracy in future cell sampling/monitoring due to sample destruction and decay; and there are concerns with contamination through swabbable valves.

There are many other methods, each of which has its own drawbacks in terms of opportunities for contamination and wastage of cell culture. It is desirable, therefore, that a device or system be developed that avoids or ameliorates on or more of the abovementioned disadvantages of existing methodologies, or at least provides a useful alternative.

### SUMMARY

In view of the abovementioned drawbacks of the prior art, disclosed is a device for aseptic small volume sampling of liquid, comprising:
a flow control system (FCS) comprising a pump; and
a tubing manifold,
wherein the tubing manifold comprises a first air point, a sample inlet for fluidly coupling the tubing manifold to a cell culture vessel, a second air point, and a sample outlet for fluidly coupling the tubing manifold to a collection point, the tubing manifold being operatively coupled to the FCS when in use, the FCS comprising a plurality of valves operable to selectively open and close the first air point, second air point, sample inlet and sample outlet,
wherein, in use, the FCS operates the pump and valves in a forward flow configuration to pump bulk fluid from the cell culture vessel into the sample inlet such that a predetermined volume of the bulk fluid is pumped to the sample outlet and out of the tubing manifold to the collection point, and a portion of the bulk fluid remains in the tubing manifold,
wherein, in use, the FCS operates the pump and valves in a reverse flow configuration to return the portion of the bulk fluid back to the cell culture vessel without exposing the portion of the bulk fluid to gas.

Also disclosed is a method for aseptic small volume fluid sampling using a tubing manifold, the tubing manifold comprising a first air point, a sample inlet fluidly coupled to a cell culture vessel, a second air point, and a sample outlet fluidly coupled to a collection point, the method comprising:
operating in a forward flow configuration by:
   pumping a bulk fluid from the cell culture vessel into the tubing manifold through the sample inlet and concurrently driving air out of the tubing manifold through the second air point,
   pumping the bulk fluid through the tubing manifold through the sample inlet, and concurrently driving air out of the tubing manifold through the sample outlet, and
   pumping gas into the tubing manifold through the first air point and concurrently driving a predetermined volume of fluid from the bulk fluid out of tubing manifold through the sample outlet, to the collection point while keeping a portion of the bulk fluid stationary in the tubing manifold, and
operating in a reverse flow configuration by pumping the sterile air into the tubing manifold through the second air point and concurrently driving the bulk fluid out of the tubing manifold through the sample inlet, back to the cell culture vessel.

Also disclosed is a method for aseptic harvesting of liquid by using a tubing manifold, wherein the tubing manifold comprises a sample inlet, an air point, a second air point and a sample outlet and a valve at the air point and sample outlet, the method comprising:
closing the valve at the sample outlet and opening the valve at the air point, pumping a predetermined volume of fluid into the tubing manifold through the sample inlet and concurrently driving air out of the tubing manifold through the air point,
closing the valve at the air point and opening the valve at the sample outlet; and
driving the predetermined volume of fluid out of tubing manifold through the sample outlet.

Also disclosed is a tubing manifold comprising a first air tube, a sample tube, a second air tube, a sample outlet tube, and a pump section fluidly connecting the tubes, wherein the tubing manifold is configured to engage a flow control system (FCS) such that each of the first air tube, second air tube, sample tube and sample outlet tube engages a respective valve of the FCS and the pump section engages a pump of the FCS.

Terms such as “air point”, “air inlet”, “air outlet”, “collection point”, “sample inlet”, “sample point”, “sample outlet (or collection point)” and others each refer to points or locations in the manifold at which air, bulk fluid or sample may enter or exit the manifold. The function of those features will be clear from the context of use rather than the specific term given to the relevant feature. For example, the flow control system can operate in a forward flow configuration or mode and a reverse flow configuration or mode, such that the term “air point” refers to a feature through which air, in the forward flow configuration, may enter the manifold and, in the reverse flow configuration, may exit the manifold. Depending on context, that same feature may be referred to as an “air inlet” or “air outlet”, without loss of generality of the functions that feature is capable of performing.

The phrases “at the sample inlet” and “at the sample outlet” can mean where the tubing manifold enters/exist the tubing housing, starts to extend away from the FCS or any other location suitable for providing the functions set out herein and as dictated by context.

The phrase “towards the sample outlet” refers to air or fluid being pumped through the tubing manifold in the direction of the sample outlet. In respect of the fluid, the predetermined volume of fluid (i.e. the sample to be collected) will not be the entirety of the fluid pumped into the tubing manifold when pumping that fluid towards the sample outlet. Instead, fluid will be pumped into the tubing manifold until the predetermined volume of fluid is located either downstream (fluid flowing from the sample point downstream to the sample outlet) of a valve that can be actuated to open and close the sample point or downstream of the pump of the fluid control system. Thus, any fluid that is upstream of the predetermined volume of fluid can be returned to the sample point by operating the fluid control system in the reverse flow configuration.

The term “gas” may refer to “air”—e.g. from the external environment, “sterile air” or any other suitable gas used for present purposes, particular where the gas maintains sterility of the bulk fluid.

### BRIEF DESCRIPTION OF THE DRAWINGS

Embodiments of the present invention will now be described, by way of non-limiting example, with reference to the drawings in which:

FIG. 1 is a top view of an aseptic sampling device in accordance with present teachings.

FIG. 2 is an exploded schematic view of an automated aseptic sampling device in accordance with present teachings.

FIG. 3 is an assembled automated aseptic sampling device with a tubing manifold located is a tubing panel (presently a single-use or disposable tubing panel), the tubing panel being locked onto a Flow Control System (FCS) thereby maintaining the tubing manifold in register with (i.e. operably engaged with) the FCS.

FIG. 4 is a bottom view of the disposable tubing panel without the tubing manifold.

FIG. 5 shows the tubing manifold.

FIG. 6 illustrates a fully assembled automated aseptic sampling device with the tubing sensors.

FIG. 7 is an example graphic user interface (GUI) for controlling an automated aseptic sampling programme performed with the aseptic sampling device of FIG. 1.

FIGS. 8 to 12 are top views of the fully assembled automated aseptic sampling device, with the disposable tubing housing hidden, in which, respectively: the bulk fluid is being drawn from the cell culture vessel to the first sensor adjacent to the sample inlet; the bulk fluid is being drawn through the tubing manifold via the sample inlet; the predetermined volume of fluid is being separated from the bulk liquid; the predetermined volume of fluid is being driven out of the tubing manifold through the sample outlet, past the second sensor, and to the collection point; and the portion of the bulk fluid remaining in the tubing manifold is being returned to the cell culture vessel.

FIG. 13 is a top view of the fully assembled automated aseptic sampling device, with the disposable tubing panel hidden, with the bulk fluid being harvested from the cell culture vessel directly to the collection point.

FIG. 14 is an exploded view of a design variation for the automated aseptic sampling device in accordance with present teachings.

FIG. 15 is an assembled view of the device of FIG. 14.

FIG. 16 is a bottom view of the tubing housing of the device of FIG. 14.

FIG. 17 is a top view of the tubing manifold for the device of FIG. 14.

FIG. 18 shows the device of FIG. 14, with attached sensors.

FIG. 19 is a top view of the device of FIG. 14, with the disposable tubing panel hidden.

### DETAILED DESCRIPTION

Disclosed are devices or systems for automated, aseptic, small volume fluid sampling. The devices enable aseptic small volumes of fluid (e.g. cell culture medium) to be sampled substantially without opportunity for contamination and substantially without loss or wastage of fluid.

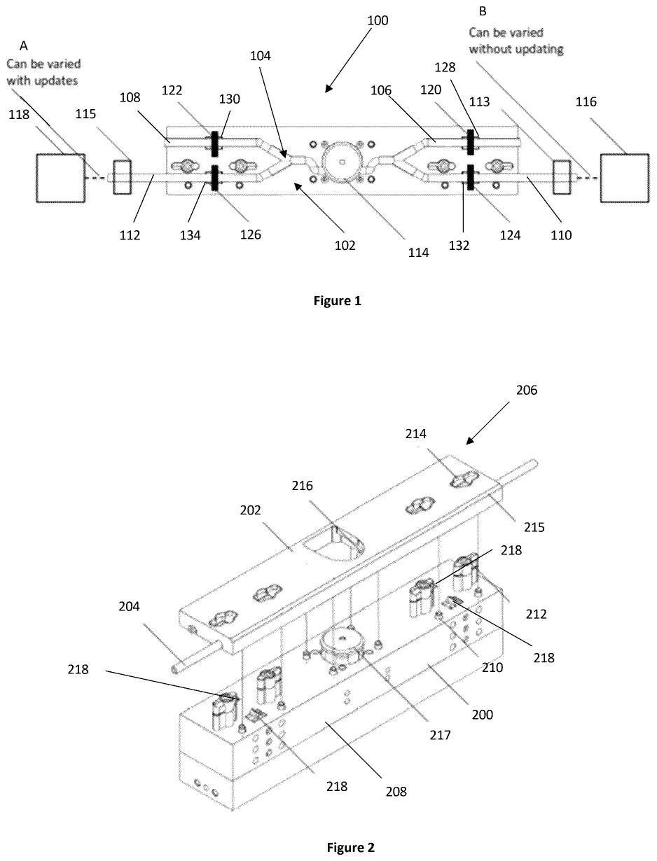

A device 100 for aseptic small volume sampling of liquid is shown in FIG. 1. As used herein, small volumes will typically be less than 10 mL, such as from 20 μl to 1 mL. The device 100 broadly includes a flow control system (FCS) 102 and a tubing manifold 104.

The FCS 102 controls the flow of fluid through the tubing manifold 104. To that extent, the FCS 102 may take any appropriate configuration to achieve the function of driving fluid through the tubing manifold 104 according to the control methodologies set out herein.

With reference to FIG. 2, in some embodiments the FCS 200 interacts with a tubing housing 202 to maintain the position of the tubing manifold 204 during use. Together, the FCS 200, tubing housing 202 and tubing manifold 204 form a Device 206 for Automated Aseptic Sampling (DAAS—shown in exploded view in FIG. 2) for sampling small volumes. The tubing housing 202 may be disposable or otherwise single-use, alone or together with the tubing manifold 204. IN this way, the FCS 200 can be reused, whereas the components that directly contact the fluid being sampled are disposable. The tubing housing 202 and tubing manifold 204 may be provided in a single—use kit that is replaced every time the DAAS 206 is used with a fresh cell culture. The single-use kit can be injection moulded from Polystyrene (PS), Polycarbonate (PC) and/or Polypropylene (PP), or any other suitable material. PS or PC may be used if a transparent finish is required. The kit (tubing housing and tubing manifold) may be produced for use with a specific cell culture—e.g. sized for a particular culture medium or cell size, to enable pumping of the cell culture without damage to cells. For packaging, the kit can be either packaged together with a cell culture vessel. The tubing housing and tubing manifold may be sterilized, for example by using gamma or beta irradiation, or ethylene oxide gas (ETO) sterilization.

The kit may further comprise sterile connecters and the cell culture vessel, to enable swift connection of the kit to the FCS prior to sampling.

The FCS 200 on the other hand is a reusable, capital device. The FCS 200 is robust to withstand repeated use. The body 208 of the FCS 200 may be made from a sterilisable material—e.g. a material that can be safely put into an autoclave (for example, steel such as 304 or 316 grade stainless steel), or cleaned with 70% ethanol, once stripped of all its electrical components.

To ensure accurate functioning of the FCS 200 in controlling fluid flow through the tubing manifold 204, the tubing manifold 204 should be precisely positioned on the FCS 200. This can be achieved by providing a groove in the FCS 200 corresponding to the shape of the tubing manifold 204, the tubing manifold 204 being held in place by friction fit, clips or any other appropriate fixing device e.g. a device enabling removal of the tubing manifold 204. However, in the embodiment shown in FIG. 2, a kit comprising the tubing manifold 204 and tubing housing 202 is used. Accurate positioning and placement of the single-use kit (202, 204) onto the FCS 200 is afforded a two-part alignment device, one part of which is disposed on the FCS 200 and the other part being disposed on the tubing housing 202 (or tubing manifold 204 where no tubing housing 202 is provided), the two parts coming together to align the tubing manifold 204 with the FCS 200. Presently, alignment is achieved using alignment pins (one of which is labelled 210) disposed on the FCS 200. Alignment may similarly be achieved by positive grooves, notches, protrusions or other device designed onto the FCS 200, with the tubing housing 202 having the corresponding holes, protrusions or notches such that a low tolerance, close alignment is achieved.

If the two-part system fixes the FCS 200 to the tubing manifold 204, then no further fastening system is required. However, in the present embodiment, the FCS 200 comprises a fastening system. The fastening system comprises turn locks (one of which is labelled 212) that pass through respective cavities (one of which is labelled 214). If the locks 212 and cavities 214 are appropriately dimensioned, there may be no need for alignment devices since the locks 212 and cavities 214 will both align and fix the tubing manifold 204 to the FCS 200. The fastening mechanism may be configured to only permit the tubing manifold 204 to be installed one way (i.e. correctly) onto the FCS 200—e.g. the cavities 214 may not align with the locks 212 if the tubing housing 202 is placed the wrong way onto the FCS 200.

Any parts of the FCS 200, attached to or integrated with the FCS 200 may be formed from a robust material to facilitate reuse. That material may be the same as that from which the FCS 200 is formed, or another material as desired.

The tubing housing 202 is configured to indicate its proper orientation on the FCS 200. Presently, that configuration comprises a chamfered edge 215, indicating the front of the tubing housing 202. This chamfered edge 215 ensures correct positioning of the pump of the FCS 200 in the corresponding pump cavity 216 of the tubing housing 202. Presently, the pump is a peristaltic pump comprising a roller head 217 that fits into the pump roller head cavity 216. This also ensure proper positioning of a pump section of the tubing manifold 204 between the tubing housing 202 and the roller head 217 of the pump with enough compression of the tubing manifold 204 to enable pumping and driving of fluid therethrough. By ensuring alignment of the tubing manifold 204 and FCS 200 in only a single orientation, this also ensures the pump drives fluid in the correct direction and the appropriate valves (if any) are opened and closed to facilitate control of air or gas, and fluid.

The tubing manifold 204 comprises a plurality of air points through which air or gas can enter and exit the tubing manifold 204, a sample inlet and a sample outlet. Control of air or gas and sample fluid through the tubing manifold 204 is managed by a plurality of valves. The present valves are pinch valves, the heads of which are labelled 218. The pinch valves may be part of the capital device (i.e. FCS 200) and should also be made to ensure it is robust to handle repeated use. The pinch valve heads 218 may be replaceable—e.g. after multiple uses to enable the pinch valves to be maintained when the heads 218 wear down over repeated compressive cycles. For the pinch valve heads 218, light yet robust materials such as polycarbonate (PC), polypropylene (PP), Polytetrafluoroethylene (PTFE), Polyamide (NYLON), can be used. These materials being manufactured via injection molding also ensures that they are cost effective and easily replaced. These plastic materials also work well with sterilization procedures using the autoclave or cleaning procedures using 70% ethanol.

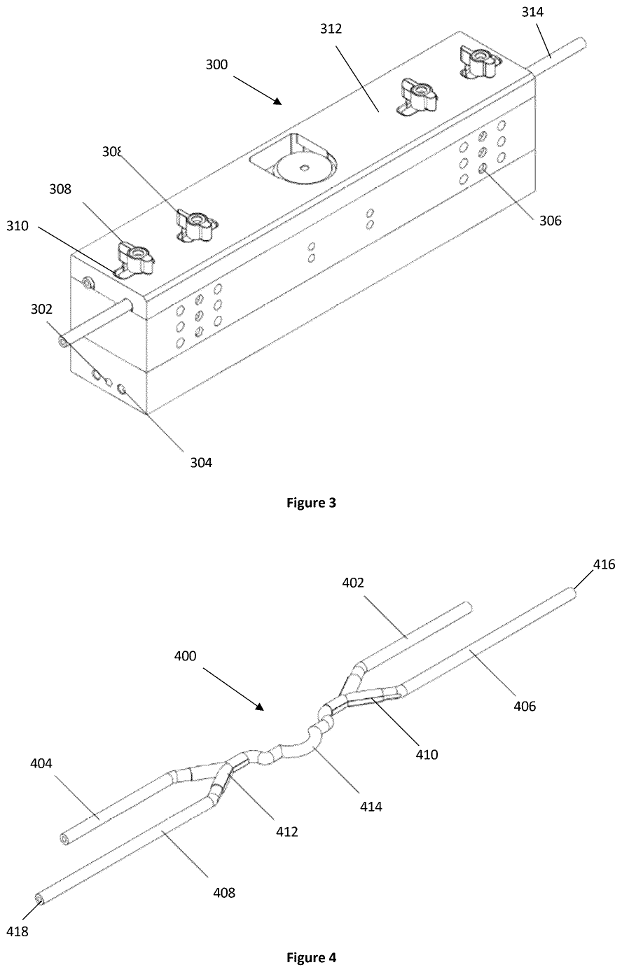

Once the tubing housing 202 and tubing manifold 204 are positioned on the FCS 200 as in FIG. 3, the alignment mechanism and/or fixation mechanism can be used to secure the assembly in its operational configuration (i.e. components are in alignment such that the FCS 200 can be operated to pump fluid through the tubing manifold 204). The turn locks 212 have been designed with internal grooves to turn through a predetermined angle of rotation—e.g. 90° —specifically in one of a clockwise or anti-clockwise direction for locking and in the opposite direction for unlocking. Thus, the locks can only be operated in a manner consistent with proper usage of the device. The alignment mechanism and/or fixation mechanism can ensure both lateral and vertical movement is prevented during use of the device (i.e. during aseptic sampling).

With reference to FIG. 3, the DAAS 300 is configured for attachment of sensors. In some embodiments, the DAAS 300 itself comprises the sensors. The DAAS 300 comprises a sensor attachment mechanism, presently magnet or magnets 302, and an alignment mechanism, presently alignment holes 304. Sensors or sensor blocks (not shown), a sensor block being a plurality of sensors in a single housing, comprise a corresponding attachment mechanism and/or alignment mechanism to facilitate attachment to the DAAS 300. Where the sensor(s) or sensor block(s) are not integrated into the DAAS 300, they may be configured for detachable attachment—e.g. through magnetic attraction to the DAAS 300, and comprising alignment pins for receipt in the alignment holes 304 thereby to align the sensor(s) or sensor block(s) on the DAAS 300. The DAAS 300 further comprises a vent (e.g. vent holes 306) for expelling heat generated during use—e.g. from a pump or from valving. Other heat dispersing systems can be used such as a heat sink and fan.

The tubing manifold 104 has a first air point 106 and second air point 108, a sample inlet 110 and a sample outlet 112. As with the embodiment shown in FIG. 2, locks 308 are inserted through alignment apertures 310 to hold the tubing housing 312 in place and thereby secure the tubing manifold 314 in its operational configuration.

An embodiment of the tubing manifold 400 is shown in FIG. 4. The tubing manifold presently comprises a first air point 402, second air point 404, sample inlet 406 and sample outlet 408.

The components 402, 404, 406 and 408 may be connected in any suitable manner to facilitate operation of the device (e.g. device 100). In the present embodiment, the tubing manifold 400 further comprises connector—e.g. Y-connectors 410, 412—for connecting the components 402, 404, 406 and 408 together. As shown in FIG. 4, the first air point 402 is connected by Y-connector 410 to the sample inlet 406, and the second air point 404 is connected to the sample outlet 408. Between the first air point 402 and sample inlet 406, and the second air point 404 and sample outlet 408 is a pump section 414. The pump section 414 aligns with the pump (e.g. the rotation head of a peristaltic pump) so the pump can drive fluid through the tubing manifold 400.

Y-connectors 410, 412 and other components (e.g. air points, inlet and outlet) of the manifold 400 can be made from polycarbonate or polypropylene, having good compatibility with gamma, beta, or ETO sterilization. Coloured polycarbonate or polypropylene can be selected to minimize effects of discolouration due to gamma irradiation. End point 416 may be fitted with a connector to facilitate connection to a cell culture vessel from which the sample can be extracted. End point 418 may similarly be fitted with a connector to facilitate connection to a monitoring system to collect and analyse samples from the said culture vessel. The connectors may each be, for example, a barbed fitting with luer connector. The culture vessel and monitoring system each have corresponding connectors—e.g. luer connectors—that can be fitted onto both ends 416 and 418 respectively. End point 418 can also be left as an open tube so that operators can collect samples into a collecting container or tube.

In addition, the entire manifold 400 can have a direct inlet line 406 going into a culture vessel, a direct output line 408 going into a monitoring system consumable set (e.g. it can be a tubing manifold set that is mounted directly onto the monitoring system), and the ends of air 402 and 404 can be connected to a gas filter housing with filter or any other necessary equipment. This entire set can be a disposable one time use only set, for a single cell culture or cell manufacturing process. The entire set can be packaged and sterilised as a single unit for example through gamma or beta irradiation, or ETO sterilisation.

Connectors may also be supplied for air points 402, 404 where needed. In each case, the connectors may be made from any suitable material such as polycarbonate or polypropylene, to enable sterilization through gamma or beta irradiation. Gas filter housings having corresponding luer fitting, with a filter (e.g. a hydrophobic filter sheet having 0.2 μm pore size) can then be fitted onto both ends 406 and 408.—The gas filters can be pre-sterilized prior to attachment to the sterilized tubing manifold, or packed together with the tubing manifold for sterilization through gamma or beta sterilization. This enables the drawing in of sterile air, and driving out of gas inside the tube, during the automated aseptic sampling process.

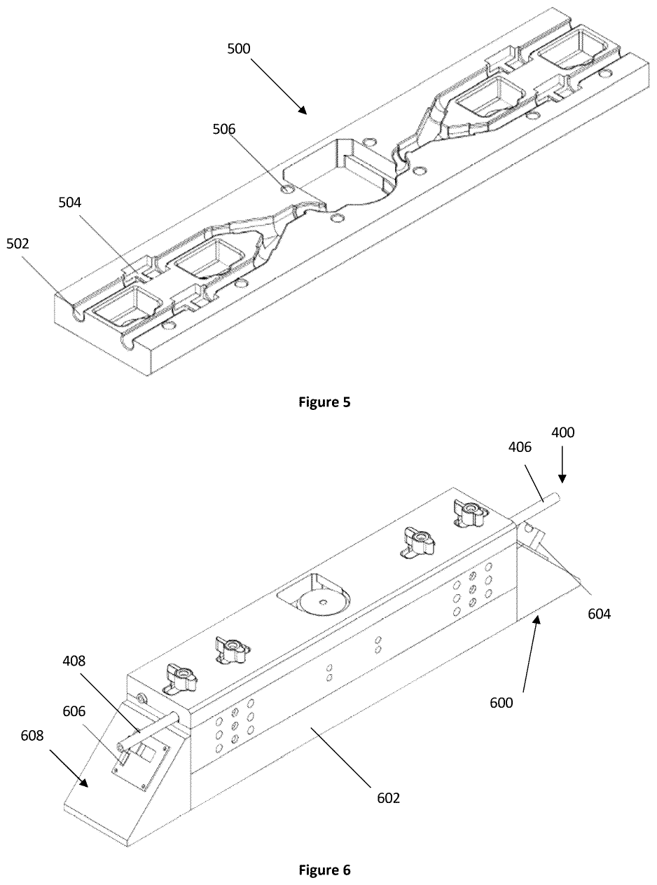

The tubing manifold 400 may be directly attached to the FCS (e.g. using tube clips or other mechanisms as described herein). The tubing manifold 400 may instead be connected to the FCS by the tubing housing 500 shown in FIG. 5. The tubing housing 500 comprises a tubing cavity or recess 502. The cavity 502 has a shape that compliments (e.g. is the negative of) the outer shape of part of the tubing manifold so that the tubing manifold is located in the tubing housing 500 with little movement. The fit between the tubing manifold and tubing housing 500 has a low tolerance fit (i.e. little, if any, room for movement) or, in the embodiment shown, a friction fit such that slight compression of the tubing manifold occurs to maintain its location in the cavity 502. With regard to the friction fit, the inner surface of the cavity 502 has a radius of curvature that is slightly smaller than the outer radius of the tubing manifold 504 to enable the tubing manifold to lodge, by friction fit, in the cavity 502 and to ensure that it does not come off loosely from the tubing housing 500. The kit may be produced in different sizes to fit on a common FCS, or to fit onto differently sized FCSs as needed. For example, the kit may comprise a tubing housing of an inner diameter from ⅛″ to 1/16″ and tubing manifold of an outer diameter from 0.25″ to 0.188″, with the sizes selected to match and maintain friction fit.

The tubing housing 500 further comprises recesses or grooves 504 for receiving valves that are operated to control fluid and airflow through the tubing manifold—e.g. pinch valves 218—when the tubing housing 500 is aligned on the FCS. The tubing housing 500 further comprises a corresponding or complementary alignment mechanism for cooperating with the alignment mechanism of the FCS, to align the FCS and tubing housing 500. Presently, the alignment mechanism of the tubing housing comprises an alignment hole 506 for each alignment pin 210.

Tubing sensors or sensor blocks 600 and 608, each sensor block comprising one or more tubing sensors (e.g. a sensor to measure a parameter of the tube, such as flow rate, whether the tube contains or does not contain fluid, fluid temperature, fluid pressure and so on), can be attached to the FCS 602 as shown in FIG. 6. Attachment may be through any appropriate means such as alignment pins (not shown) that are attracted to magnet or magnets 302 disposed in alignment holes 304. Thus, the alignment pins are magnetic, or are magnets themselves, to facilitate attraction to magnets 304. In other embodiments, the alignment pins are not magnetic. As such, the alignment pins and magnet or magnets are separate, such that alignment pins ensure proper alignment of each sensor block with the FCS, with the magnets maintaining engagement between each sensor block and the FCS. In yet further embodiments, each sensor block may have magnets that attract to the FCS, and other configurations of alignment and attachment as will be apparent to the skilled person in view of present teachings.

Sample inlet 406 is attached to sensor 604 of sensor block 600 to sense the presence of fluid in the sample inlet 406 of the tubing manifold 400—e.g. for fluid coming into the tubing manifold 400 through the sample inlet 406. Sample outlet 408 is attached to sensor 606 of sensor block 608 to sense the presence of fluid in the sample outlet 408—e.g. for fluid leaving the tubing manifold 400 through the sample outlet 408. The sensors can also be configured for different tubing sizes.

Tubes are selected to cooperate with the peristaltic pump (as used herein the term “cooperate” refers to components being shaped, sized and/or configured to engage each other to facilitate operation of the device), and enable the valves to control (along with the pump) fluid and gas/air flow through the tubing manifold. The tubing manifold may be formed from any suitable material, such as puriflex, c-flex, and ultra-c line materials. The tubing manifold may be entirely clear, or clear in areas that are visible when the tubing housing and tubing manifold are fixed to the FCS, to assist the sensors 604, 606 in identifying fluid in the tubing manifold. Materials may similarly be used that have minimal discolouration from sterilization—e.g. gamma or beta sterilization—to minimise disruption to the sensor operation.

With further reference to FIG. 1, the sample inlet 110 may be pre-connected to the cell culture vessel 116, prior to sterilization or connected separately post-sterilization, through an aseptic connector on one or both (for a two-part aseptic connector 113) of the cell culture vessel 116 and sample inlet 110. The sample outlet 112, may similarly be pre-connected to the collection point 118, prior to sterilization or connected separately post-sterilization, with both ends pre-connected with an aseptic connector 115. Pinch valve heads 120, 122, 124, 126, are connected respectively to pinch valves 128, 130, 132, 134 and operably coupled to the sample inlet 110, first air point 106, sample outlet 112, and second air (which includes gas) point 108. By thus positioning each valve, the FCS can direct the cell culture fluid and air flows and generate a sample bubble.

Information from the sensors described with reference to FIG. 6 is used by the controller to control the liquid flow. As a result, some sections of the tubing lengths can be varied without the need to re-programme the controller. Assuming the sensors are adjacent (or otherwise fixed/known distance from) the tubing housing or FCS: FIG. 1 indicates (at location s A and B) that the tubing length from the first sensor to the cell culture vessel 116 can be varied without the need to re-programme the controller whereas the tubing length from the second sensor to the collection vessel 118 needs to be updated if it was changed. Varying the tubing length, or minimising the tubing length, reduces exposure of the cell culture fluid, that was not taken in the sample, to conditions outside of the cell culture vessel.

During use, the valves 120, 122, 124, 126 may start in the closed condition, where the valve heads 128, 130, 132, 134, compress the tubing manifold 104 against the recesses or grooves 504 on the housing 500, sealing the internal cavity of the tubing manifold 104. This prevents air/gas and fluid from passing into or out of the tubing manifold 104. The collection point 118 can be a series of bags, a container manifold or other appropriate sample collection device, or a measurement or inspection system to which the device 100 is directly connected, or left open to the atmosphere where the user can collect the outgoing fluid using tubes or petri dishes.

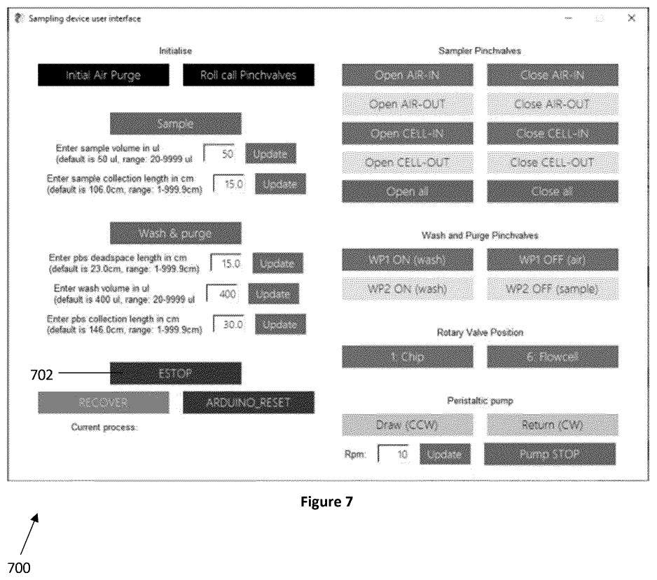

FIG. 7 shows an example GUI 700 for controlling DAAS (device 100). In addition to enabling programming of the FCS for subsequent, automated operation, the GUI 700 enables emergency stop and reset of the sampling process. The FCS may accept commands using any appropriate mechanism and protocol. Presently, the FCS accepts commands via serial communications such that the device is compatible as an integrated sampling unit within automated solutions. In an example, the controller of the sampling device is written in C/C++ programming language, is deployed via an Arduino microcontroller, and the GUI 700 is written in Python and deployed on laptop. The Python interface communicates with the Arduino microcontroller via USB serial communication. The GUI 700 also enables manual control of pinch valves and peristaltic pump. It enables users to change parameters such as tubing lengths, sample volumes, and pump speed. The default values for these parameters can be set in a configuration file if the parameters are not expected to change for a specific application.

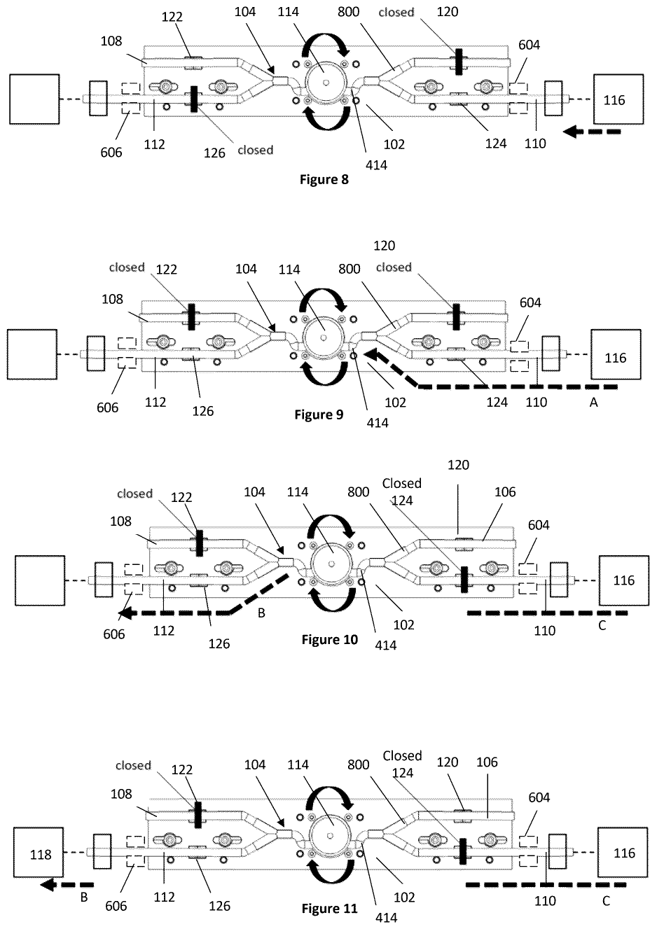

FIGS. 8 to 12 illustrate the aseptic sampling process using a device 100 in accordance with present teachings. Once the device, sample vessel and sample collection point are in operational configuration, the process can start. Initially, as shown in FIG. 8, the FCS 102 opens the valves 124 and 122 (pinch valves will be used for illustration purposes only, where other valves may be used as appropriate, whether those other valves form part of the FCS, tubing housing or tubing manifold). Concurrently, or shortly after, opening valves 122, 124 the FCS operates peristaltic pump 114 to rotate in a clockwise direction, while keeping valves 120, 126 closed. The bulk fluid in the cell culture vessel 116 is drawn into the tubing manifold 104 to the first tubing sensor 604 of the DAAS 100 while air is concurrently being driven out of the second air point 108. The fluid sensor 604 detects the bulk fluid in the tubing of inlet 110 and the FCS 102 records the time it took for the bulk fluid to travel from the cell culture vessel 116 to the first sensor 604.

The bulk fluid travels into the DAAS 100, through the sample inlet 110 of the tubing manifold 104. As it reaches the end of the first Y-connector 800, the FCS 102 determines that the bulk liquid has travelled the predetermined length based on the programme in the FCS 102, to reach the transition point (as indicated by the arrow A-i.e. a position at which the predetermined volume of fluid can now be driven by the pump, to the sample outlet, with air progressively occupying the tubing manifold as the predetermined volume of fluid progressively exits the sample outlet) from the sample inlet 110 to the sample outlet 112, and closes pinch valve 122 for the second air point 108, while opening the pinch valve 126 for sample outlet 112, as shown in FIG. 9. The pump 114 continues to turn in the clockwise direction, pumping bulk fluid into the pump section 414 of the tubing manifold 104, while driving air out of the sample outlet 112.

After the bulk fluid has travelled a predetermined distance (per arrow B-this predetermined distance can be calculated from flow rate determined by full and partial revolutions of the head of pump 114 after fluid is detected in the sample inlet 110, and the dimensions of the tubing manifold 104 when in the operational configuration), the FCS 102 determines that the respective predetermined sample volume has been achieved. In this sense, the predetermined distance is interchangeable with a predetermined volume being pumped past a point at which it cannot return to the sample vessel 116. Thus, the predetermined volume has been pumped into the manifold 104 to a position at which it can be driven out of the sample outlet 112 by sterile air (or gas) entering the tubing manifold 104 through the first air point 106. Moreover, all bulk fluid other than the predetermined volume can be displaced by air back through the sample point 110 to the cell culture vessel 116—e.g. only the predetermined volume has moved past the first Y-connector 800 as illustrated in FIG. 10. This also involves closing pinch valve 124, and opening pinch valve 120. The pump 114 continues rotating in the clockwise direction, causing the fluid from the sample pinch valve 124 to separate from the bulk fluid (indicated by line C), as a pre-determined sample volume or better known as a sample bubble (B), and driven out of the DAAS 100 through the sample outlet 112, facilitated by the pumping of sterile air from the first air point 106. This happens while a portion of the bulk fluid (C) remains stationary at the sample inlet 110 and is sealed by the sample inlet valve 124. Sealing valve 124 helps prevent contact of the portion of the bulk liquid (C) with the sample outlet 112, or collection point 118, which con-tributes to maintaining its sterility.

Once the sample bubble (B) reaches the second sensor 606 adjacent to the exit of the DAAS 100 along the sample outlet 112, the FCS 102 determines the predetermined tubing length that the sample bubble has to travel to reach the collection point 118. The pump 114 continues rotating in the clockwise direction, to completely drive the sample bubble B out of the tubing manifold 104, to the collection point 118.

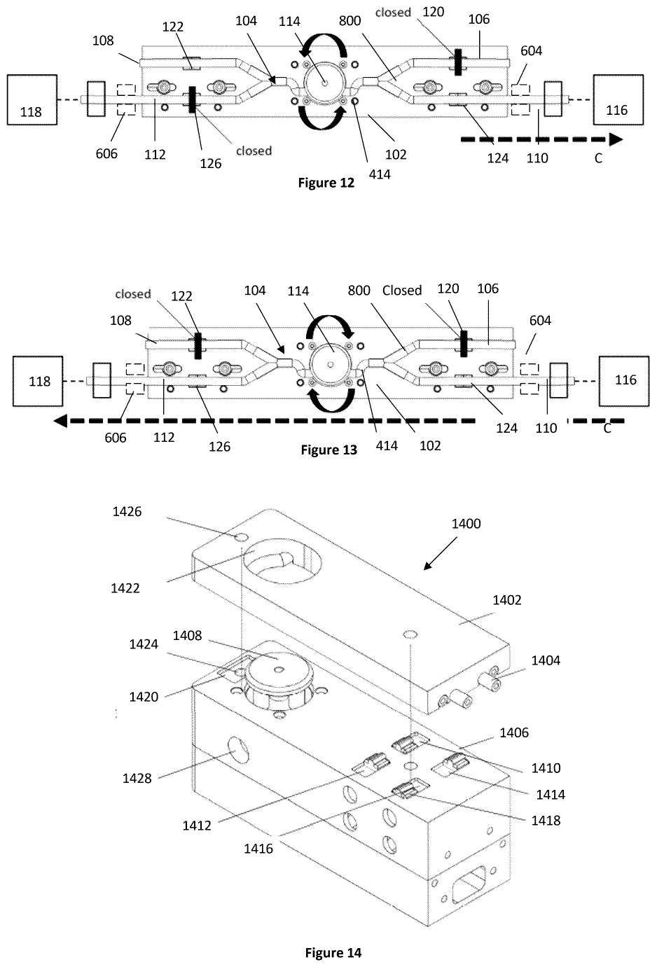

The FCS 102 thus drives the sample bubble to the collection point 118. Dispens-ing of the full sample bubble into the collection point 118 can be determined by detecting when the last fluid from the sample bubble (B) passes sensor 606, indicating that the whole sample (B) is now downstream (i.e. towards the collection point 118) of the valve 126 and of the sensor 606. The program in the FCS 102 takes into account the length of tubing at the sample outlet 112 and thus operates the pump 114 until the full sample has been delivered. Thus, the FCS 102 determines that the sample bubble (B) has completely reached the collection point 118, as illustrated in FIG. 12.

The FCS 102 then activates the reverse flow configuration. In the reverse flow configuration the return of the sterile portion of the bulk fluid (C) to the cell culture vessel 116. This is done by closing the valves 120, 126 at the sample outlet 112 and the first air point 106, and opening the valves 122, 124 at the sample inlet 110 and second air point 108 as shown in FIG. 12. This is then followed by turning the peristaltic pump 114 in the reverse configuration—i.e. in an anti-clockwise manner. This event causes the sterile portion of the bulk fluid (C) to be pumped from the sample inlet 110, to the cell culture vessel 116, while sterile air is drawn into the tubing manifold 104 through the second air point 108. This prevents any fluid or contaminated air from the collection point, from being pumped back into the tubing manifold 104, potentially causing contamination to the bulk fluid. When the bulk fluid passes the first sensor, the FCS 102, uses the recorded time that it took for the bulk fluid to travel from the cell culture vessel 116 to the first sensor 604, to return the portion of the bulk fluid (C) completely to the cell culture vessel 116. Once the FCS 102 determines, based on the recorded time, that there is no longer fluid in the tubing manifold, it stops the pump 114, and closes all the pinch valves 120, 122, 124, 126, returning to the original configuration in FIG. 1.

In another configuration, the DAAS 100 has been configured to perform the harvesting process of the entire bulk fluid from the cell culture vessel 116 to the collection point 118 as illustrated in FIG. 13. In this instance, valves 124, 126 are opened while valves 120, 122 are closed, and the pump 114 is operated until fluid from the sample vessel 116 has passed sensor 606 and keeps operating until no further fluid is detected by sensor 606, plus a margin for pumping the fluid through the full length of the sample outlet 112. This is done only if the collection point 118 already has a separate sterile and sealed tubing manifold (not shown) with a sterile connector for connecting of a sterile collection bag or the collection bag has already been pre-connected to the tubing manifold 104. The portion of the tube previously used for sampling to sampling bags on the tubing manifold, should be sealed off permanently after every sampling procedure to prevent any contamination from the sampling bags from potentially contaminating the tubing manifold. If the sampling collection point 118 is exposed to air, there needs to be another clamp or valve between this sample collection point 118, and the point leading to the harvest bag and the second sensor. This ensures that the sample collection point 118 can always be sealed off after every collection of the sample bubble, together with closing the valve 126 at the sample outlet, to prevent any contamination of the harvest bag or the tubing leading from the second sensor to the harvest bag. This can also be done if the collection point 118 is already connected to a measurement system which can ensure the bulk fluid collected remain sterile, and is able to sort this harvested bulk fluid in a sterile fashion.

Valves 124, 126 are opened while the valves 120, 122 remain closed. The pump 114 is then turned in the forward clockwise direction to pump the bulk fluid from the cell culture vessel 116 to the collection point 118. The second sensor 606, determines when the bulk fluid has exited the housing, and the FCS 102 determines the pre-determined tubing length it must travel to reach the harvest collection bag at 118.

If there is an issue with the DAAS 100 or/and the sampling function or/and the harvesting function, the emergency stop button can be pressed. When the emergency button is pressed, the device closes all pinch valves and stops the peristaltic pump. All other functions are disabled until the system is reset.

When reset button is pressed during emergency stop 702, the cell culture in the cell culture inlet tubing section 110 is returned to the cell culture vessel 116. The cell culture inlet pinch valve 124 and second air point pinch valve 122 are opened and the peristaltic pump 114 is set to rotate anti-clockwise. In the event that the fluid has not reached the first fluid sensor 604, the time from start of routine till the time when emergency stop is activated is recorded. The pump 114 rotates anti-clockwise for the time period recorded. In the event that the fluid has passed the first fluid sensor 604, the fluid sensor 604 is used to detect the return of the cell culture.

The sample in the cell sample outlet tubing section 112 is then purged. The cell culture inlet pinch valve 124, and valve 122 are closed. Then, the valve 120, and cell sample outlet pinch valve 126 are opened, and the peristaltic pump 114 is set to rotate clockwise for a set period of time to fully empty the tubing section. Once the FCS 102 has determined that all the fluid has been removed from the tubing manifold 104 (e.g. pumping for a predetermined period of time), all the pinch valves are closed, and the peristaltic pump is stopped.

FIG. 14 is an exploded view of a miniaturized DAAS 1400. Device 1400 is smaller from the DAAS 100 by at least half the length. Configuration and operation for automated aseptic sampling remains the same as for device 100. Device 1400 still consists of a, potentially single-use, kit comprising the tubing housing 1402 and tubing manifold 1404, that are coupled in use to the FCS 1406. The pump 1408 has been repositioned to one end of the DAAS 1400 while the pinch valves 1410, 1412, 1414, 1416 for sample inlet, sample outlet, first air point, and second air point, has been collected and positioned to the other end of the DAAS. The pinch valve heads (one of which is identified by 1418) for the pinch valves have also been redesigned for this configuration. Each head 1418 enables the pair of tubes, either sample inlet and first air point, or sample outlet and second air point, to go through each pinch valve together, while only one tube of the pinch valve is engaged and disengaged by the pinch valve at their respective location. Each pinch valve therefore has a first condition in which it engages a first tube (one of the air points, sample inlet and sample outlet) and a second condition in which it engages a second tube (one of the air points, sample inlet and sample outlet). The pinch valves may move in any desired manner—for example, the pinch valves may move vertically up and down in a height direction of the FCS (i.e. towards and away from tubing housing 1402), between the first and second conditions. For instance, the sample inlet tube will be engaged or disengaged by the pinch valve head 1418 and its corresponding pinch valve 1410, even though both sample inlet tube and first air point tube go through the same pinch valve 1410. This enables a more compact redesign of the DAAS 1400 while keeping the fundamentals of the operations and configurations the same.

The negative alignment groove 1420 has been designed to both locate and align the kit (1402, 1404) onto the FCS 1406, and lock it against lateral movements. Movements may occur, for example, from peristaltic pump 1408 rotating motion against the tube 1404 in between the pump and the curved pump cavity 1422 in the tubing housing 1402. Hole 1424 in the negative groove 1420 has been threaded, to enable the use of knurled or knob screws to secure and lock the single-use kit through the hole 1426, onto the FCS 1406. The screws, act par-tially to lock the single-use kit against lateral movements, but primarily to lock it against vertical movements, coming from the pinch valves 1410, 1412, 1414, 1416. The location and design of the grooves and screws were intentional to enable the compact design of DAAS 1400. In addition with the miniaturized design, less securing and locating parts were needed, reducing the number of parts and steps that the user has to focus on while assembling the DAAS 1400. The FCS 1406 still consists of venting holes 1428 for the peristaltic pump 1408 and the pinch valves 1410, 1412, 1414, 1416 to keep these components cool under prolonged and repeated operations.

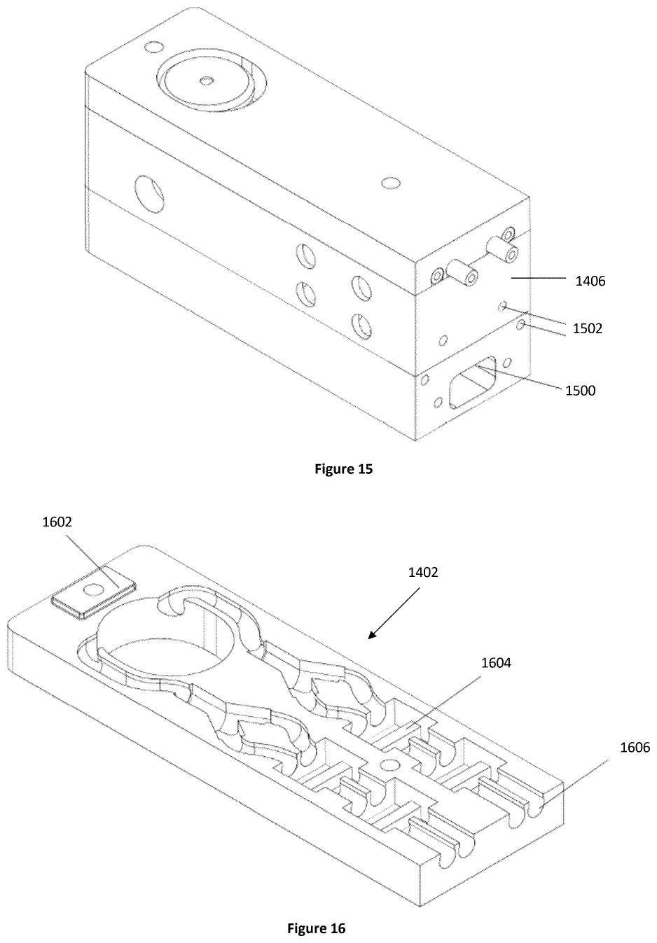

The fully assembled miniaturized DAAS 1400 in FIG. 15 comprises a single cable hole 1500 for internal components like the peristaltic pump and the pinch valves. It also consists of aligning threaded holes 1502 for the sensor block attachment.

The tubing housing 1402, in FIG. 16, comprises a positive alignment groove or projection 1602, for mating with the negative alignment groove 1420 to lock the kit onto the FCS 1406, against lateral movements. The pinch grooves 1604, align with the pinch valve head 1418, to pinch or un-pinch the tube 1404 located in between. The tube cavity 1606 is the same as for the device 100.

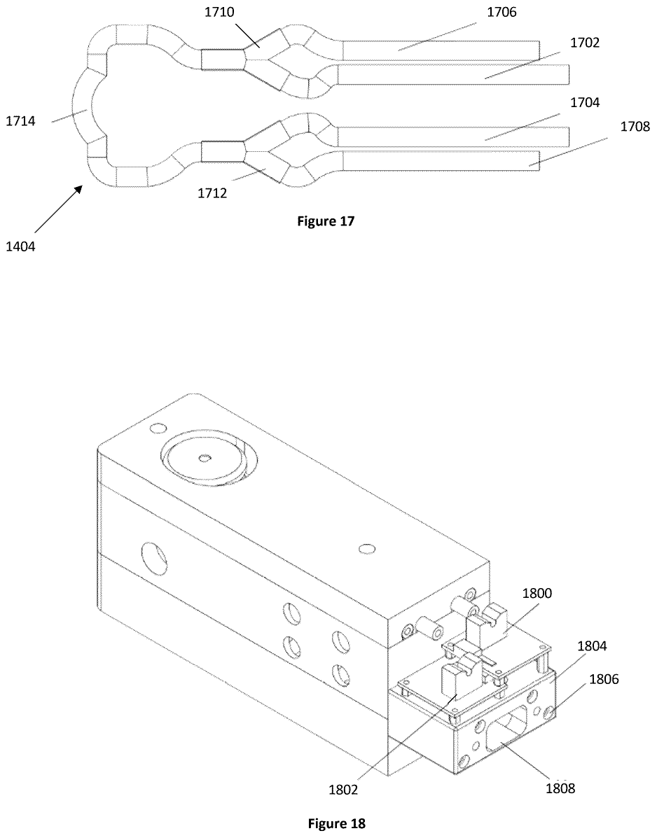

The tubing manifold 1404 in FIG. 17, has substantially the same features as the tubing manifold 104 (i.e. in substance, such that the operation is the same though the particular arrangement is modified). The manifold 1404 comprises a sample inlet 1702 and sample outlet 1704, and first air point 1706 and second air point 1708. The manifold 1404 also comprises y-connectors 1710, 1712 for both pairs of tubes (1706 & 1702, and 1704 & 1708), and a pump section 1714. If the tubing manifold in FIG. 17 is formed into a U-shape, with the sample outlet 1704 and the second air point 1708 facing the same direction to sample inlet 1702 and the first air point 1706, when compared with the straightened embodiment in which the sample inlet and first air point face the opposite direction to the sample outlet and second air point, per Figure, it would look similar to the original tubing manifold in FIG. 4.

The FIG. 18 shows the device 1400 of FIG. 14 in a fully assembled condition, using tubing housing 1402 of FIG. 16 and manifold 1404 of FIG. 17, along with a first sensor 1800 and second sensor 1802. The sensors 1800, 1802 may be directly affixed to the FCS 1406 or may, as shown, be incorporated into a sensor block 1804 that is then attached to the FCS 1406 in a manner consistent with sensor block 600 of FIG. 6. The sensor block further comprises an attachment mechanism, presently being screw holes 1806 through which screws can be inserted into holes 1502 of the FCS 1406 to attach the sensor block 1804 thereto. The sensor block 1804 comprises a cavity 1808 for a cable connected to both sensors 1800, 1802.

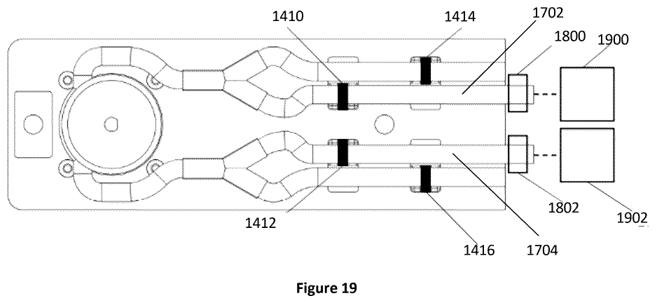

The tubing manifold 1404 and tubing housing 1402 in FIG. 19 are connected in a similar manner to those shown in FIG. 1, to the cell culture vessel 1900 through the sample inlet 1702, and to the collection point 1902, through the sample outlet 1704. The first sensor 1800 is located on the tubing of the sample inlet 1702, adjacent to the entrance into the tubing manifold 1404 within the kit (i.e. tubing housing 1402 and tubing manifold 1404, which may together form a disposable or single-use unit). The second sensor 1802, is located on the tubing of the sample outlet 1704, adjacent to the exit of the tubing manifold 1404. Operation of the device 1400 (with tubing housing 1402 and tubing manifold 1404, mounted to FCS 1406) is the same as that described with reference to FIGS. 8 to 13.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A device for aseptic small volume sampling of liquid, comprising:
a flow control system (FCS); and
a tubing manifold,
wherein the tubing manifold comprises an air point 1, a sample inlet fluidly coupled to the cell culture vessel, an air point 2, and a sample outlet fluidly coupled to the collection point, each of which is fluidly coupled to the tubing manifold, and operatively coupled to the FCS,
wherein the FCS is operable in a forward flow configuration to pump bulk fluid from the cell culture vessel to the sample inlet on the tubing manifold,
wherein the FCS is operable in a forward flow configuration to pump a predetermined volume of fluid, from said bulk fluid, while keeping the bulk fluid stationary, from the sample inlet to the sample outlet, and out of the tubing manifold, to the collection point,
wherein the FCS is operable after pumping the predetermined volume of fluid out of the sample outlet and tubing manifold, in a reverse flow configuration to return the bulk fluid remaining in the sample inlet on the tubing manifold, back to the cell culture vessel.
wherein said forward and reverse configurations and operations maintains sterility of the bulk fluid in the cell culture vessel,
wherein said forward and reverse configurations and operations prevents dead volume of fluid in any part of the tubing from the cell culture vessel to the tubing manifold, and to the collection point,
wherein said forward flow and reverse flow configurations and operations prevent air bubble formation in fluid exiting the collection point or bulk fluid returning to the cell culture vessel.

2. The device of 1, wherein the predetermined volume of fluid is a sample bubble of a programmable volume.

3. The device of 2, wherein the programmable volume is consistent through repeated sampling process.

4. The device of 2 or/and 3, wherein the programmable volume is ranging from 20 μl to 1 ml.

5. The device of 1, wherein the FCS comprises a valve at each of the air point 1, sample inlet, air point 2, and sample outlet, and a pump.

6. The device of 5, wherein the FCS being configured to control the valves and actuate the pump to draw the bulk fluid from the cell culture vessel into the tubing manifold through the sample inlet and concurrently drive the air out of the tubing manifold through the air point 2.

7. The device of 5 and 6, wherein the FCS opens the valve at the sample inlet and air point 2, closes the valve at sample outlet and air point 1, and actuates the pump to rotate in a first direction when drawing the bulk fluid from the cell culture vessel into the tubing manifold through the sample inlet and concurrently driving the air out of the tubing manifold through the air point 2.

8. The device of 5 and 7, wherein the FCS being configured to control the valves and actuate the pump to draw the bulk fluid from the cell culture vessel through the manifold, through the sample inlet and concurrently drive the air out of the tubing manifold through the sample outlet.

9. The device of 5 and 8, wherein the FCS opens the valve at the sample inlet and sample outlet, closes the valve at air point 1 and air point 2, and actuates the pump to rotate in a first direction when drawing the bulk fluid from the cell culture vessel through the tubing manifold, through the sample inlet and concurrently driving the air out of the tubing manifold through the sample outlet.

10. The device of 5 and 9, wherein the FCS being configured to control the valves and actuate the pump to draw sterile air into the tubing manifold through the air point 1 and concurrently drive a predetermined volume of fluid from the bulk fluid, while keeping the bulk fluid stationary, out of the manifold, through the sample outlet, and to the collection point.

11. The device of 5 and 10, wherein the FCS opens the valve at the air point 1 and sample outlet, closes the valve at sample inlet and air point 2, and actuates the pump to rotate in a first direction when drawing sterile air into the tubing manifold through the air point 1 and concurrently driving a predetermined volume of fluid from the bulk fluid, while keeping the bulk fluid stationary, out of the tubing manifold, through the sample outlet, and to the collection point.

12. The device of 5 and 11, wherein the FCS being configured to control the valves and actuate the pump to draw sterile air into the tubing manifold through the air point 2 and concurrently driving the bulk fluid out of the manifold, through the sample inlet, back into the cell culture vessel.

13. The device of 5 and 12, wherein the FCS opens the valve at the air point 2 and sample inlet, closes the valve at sample outlet and air point 1, and actuates the pump to rotate in a second direction opposed to the first direction when drawing sterile air into the tubing manifold through the air point 2 and concurrently driving the bulk fluid out of the manifold, through the sample inlet, back into the cell culture vessel.

14. The device of 5, wherein the FCS being configured to control the valves and actuate the pump to draw bulk fluid from the cell culture vessel into the tubing manifold through the sample inlet and concurrently driving the bulk fluid out of the manifold, through the sample outlet, and to the collection point.

15. The device of 5 and 14, wherein the FCS opens the valve at the sample inlet and sample outlet, closes the valve at air point 1 and air point 2, and actuates the pump to rotate in a first direction when drawing bulk fluid from the cell culture vessel into the tubing manifold through the sample inlet and concurrently driving the bulk fluid out of the manifold, through the sample outlet, and to the collection point.

16. The device of 1, wherein the FCS comprises a first sensor positioned adjacent the sample inlet and a second sensor positioned adjacent the sample outlet, wherein the first sensor and the second sensor are configured to detect presence of fluid at the sample inlet and sample outlet, respectively.

17. The device of 16, wherein the FCS is configured to control the forward and reverse flow configurations and operations in accordance with detection of fluid from the first and second sensors to ensure volume consistency of the predetermined volume of fluid.

18. The device of 16, wherein the FCS is configured to control the forward and reverse flow configurations and operations in accordance with detection of fluid from the first and second sensors to ensure the sterility of the bulk fluid.

19. The device of 16, wherein the FCS is configured to control the forward and reverse flow configurations and operations in accordance with detection of fluid from the first and second sensors to prevent the formation of bubbles in the predetermined volume of fluid exiting the sample outlet or bulk fluid returning to the cell culture vessel.

20. The device of 16, wherein the FCS is configured to control the forward and reverse flow configurations and operations in accordance with detection of fluid from the first and second sensors to prevent dead volume in any part of the tubing, from the cell culture vessel to the tubing manifold and to the collection point.

21. The device of 16, the first sensor being configured to detect when the bulk fluid enters the tubing manifold via the sample inlet, the FCS determining when the bulk fluid with volume has entered the tubing manifold via the sample inlet.

22. The device of 21 when dependent on 6, the FCS opens the valve on the sample inlet and the air point 2, closes the valve on the air point 1 and the sample outlet, and actuates the pump to rotate in the first direction to draw the bulk fluid from the cell culture vessel to the first sensor adjacent to the sample inlet, and concurrently drive the air out of the tubing manifold through the air point 2.

23. The device of 22, wherein the FCS records the amount of time it took to draw the bulk fluid from the cell culture vessel to the first sensor.

24. The device of 22 when dependent on 8, the FCS opens the valve on the sample inlet and the sample outlet, closes the valve on the air point 1 and the air point 2, and actuates the pump to rotate in the first direction when the FCS determines that the bulk fluid has reached the predetermined transition point, which is based on the predetermined distance from the first sensor to the predetermined transition point from the sample inlet to the sample outlet.

25. The device of 24 when dependent on 8, the FCS opens the valve on the sample inlet and the sample outlet, closes the valve on the air point 1 and the air point 2, and actuates the pump to rotate in the first direction, to draw the bulk fluid through the tubing manifold through the sample inlet, and concurrently drive the air out of the tubing manifold through the sample outlet.

26. The device of 25 when dependent on 10, the FCS opens the valve on the air point 1 and the sample outlet, closes the valve on the air point 2 and the sample inlet, and actuates the pump to rotate in the first direction when the FCS determines that the predetermined volume of fluid has been achieved from when the bulk fluid was first detected at the first sensor.

27. The device of 26 when dependent on 10, the FCS opens the valve on the air point 1 and the sample outlet, closes the valve on the air point 2 and the sample inlet, and actuates the pump to rotate in the first direction when drawing sterile air into the tubing manifold through the air point 1 and concurrently driving the predetermined volume of fluid from the bulk fluid, while keeping the bulk fluid stationary, out of the tubing manifold, through the sample outlet.

28. The device of 26 and 16, the second sensor being configured to detect when the predetermined volume of fluid exits the tubing manifold via the sample outlet, the FCS determining when the predetermined volume of fluid has exited the tubing manifold via the sample outlet.

29. The device of 28 when dependent on 10, the FCS opens the valve on the air point 1 and the sample outlet, closes the valve on the air point 2 and the sample inlet, and actuates the pump to rotate in the first direction, when the second sensor detects the predetermined volume of fluid, the FCS determining the predetermined length of tubing for the predetermined volume of fluid to travel from the second sensor to the collection point.

30. The device of 29 when dependent on 10, the FCS opens the valve on the air point 1 and the sample outlet, closes the valve on the air point 2 and the sample inlet, and actuates the pump to rotate in the first direction when drawing sterile air into the tubing manifold through the air point 1 and concurrently driving the predetermined volume of fluid from the bulk fluid, while keeping the bulk fluid stationary, from the second sensor to the collection point.

31. The device of 30 when dependent on 12, the FCS opens the valve on the air point 2 and the sample inlet, closes the valve on the air point 1 and the sample outlet, and actuates the pump to rotate in the second direction opposed to the first direction, when FCS has determined that the predetermined volume of fluid has reached the collection point.

32. The device of 31 when dependent on 12 and 23, the FCS opens the valve on the air point 2 and the sample inlet, closes the valve on the air point 1 and the sample outlet, and actuates the pump to rotate in the second direction opposed to the first direction, when drawing sterile air into the tubing manifold through the air point 2 and concurrently driving the bulk fluid out of the manifold, through the sample inlet, back into the cell culture vessel.

33. The device of 32 dependant on 23, the FCS being configured to determine when the bulk fluid has been completely returned to the cell culture vessel, and there is no more fluid in the tubing from the cell culture vessel to the tubing manifold, and to the collection point.

34. The device of 33, the FCS closes the air point 1, air point 2, sample inlet, and sample outlet, and stops the pump, on determining that there is no fluid in the tubing from the cell culture vessel to the tubing manifold, and to the collection point.

35. The device of any one of 1, wherein the collection point is connected to a series of sample bags or container manifolds.

36. The device of 35, wherein the reverse flow configuration comprises returning bulk fluid that is untouched by the collection point, or the sample bags or container manifolds.

37. The device of 36, wherein the reverse flow configuration comprises returning bulk fluid that has been separated from the sample bubble, to the cell culture vessel.

38. The device of any one of 1, 2, and 37, wherein the sample bubble created out of the bulk cell culture fluid and is separated from bulk cell culture fluid.

39. The device of 38, wherein the FCS is configured to seal (e.g. by closing the valve at the sample inlet/sample point) the bulk cell culture fluid from exposure to contamination at the collection point.

40. The device of 38, wherein the FCS pumps the sample bubble out of the tubing manifold through the sample outlet, to the collection point, once the sample bubble is created.

41. The device of any of 1, wherein the collection point is exposed to the atmosphere.

42. The device of any of 1, wherein the collection point is flexibly adapted to measurement systems.

43. The device of any of 1, wherein the tubing manifold is carried in a disposable housing which is attachable to the FCS.

44. The device of 43, wherein the housing comprises aligning grooves or pins and the FCS comprises the respective mating holes or notches for the grooves or pins, that enable the housing to be located on the FCS and locked against lateral movement, where the tubing manifold in the disposable housing is then operably coupled to the peristaltic pump and pinch valves of the FCS.

45. The device of 44, wherein the FCS comprises threaded holes and the housing comprises the respective mating non-threaded holes, that enable the housing, to be located on the FCS and to be locked against lateral and vertical movement, by screws or turn locks.

46. The device of 43, wherein the tubing manifold is located between the housing and either the pinch valves or the peristaltic pump of the FCS.

47. The device of 46 wherein the housing comprises grooves that align with the pinch valves, when assembled to the FCS, for enabling pinching and unpinching of the tubing manifold.

48. The device of 47, wherein the housing comprises a curved wall that align with the peristaltic pump, when assembled to the FCS, for enabling pumping of fluid in the tubing manifold.

49. A method for aseptic small volume sampling of liquid by using a tubing manifold, wherein the tubing manifold comprises an air point 1, a sample inlet fluidly coupled to the cell culture vessel, an air point 2, and a sample outlet fluidly coupled to the collection point, each of which is fluidly coupled to the tubing manifold the method comprising:
pumping a bulk fluid from the cell culture vessel into the tubing manifold through the sample inlet and concurrently driving air out of the tubing manifold through the air point 2,
pumping a bulk fluid through the tubing manifold through the sample inlet, and concurrently driving air out of the tubing manifold through the sample outlet,
pumping the sterile air into the tubing manifold through the air point 1 and concurrently driving a predetermined volume of fluid from the bulk fluid, while keeping the bulk fluid stationary, out of tubing manifold through the sample outlet, to the collection point and,
pumping the sterile air into the tubing manifold through the air point 2 and concurrently driving the bulk fluid out of the tubing manifold through the sample inlet, back to the cell culture vessel.

50. The method of 49, wherein pumping of the bulk fluid from the cell culture vessel into the tubing manifold through the sample inlet and concurrently driving the air out of the tubing manifold through the air point 2, comprises of closing the air point 1 and sample outlet, and opening of the air point 2, and sample inlet.

51. The method of 49 wherein the pumping of the bulk fluid through the tubing manifold through the sample inlet, and concurrently driving air out of the tubing manifold through the sample outlet, comprises of closing the air point 1 and air point 2, and opening of the sample inlet and sample outlet.

52. The method of 49 wherein the pumping of the sterile air into the tubing manifold through the air point 1 and concurrently driving a predetermined volume of fluid from the bulk fluid, while keeping the bulk fluid stationary, out of tubing manifold through the sample outlet, to the collection point, comprises of closing the air point 2 and sample inlet, and opening of the air point 1 and sample outlet.

53. The method of 49 wherein the pumping of the sterile air into the tubing manifold through the air point 2 and concurrently driving the bulk fluid out of the tubing manifold through the sample inlet, back to the cell culture vessel, comprises of closing the air point 1 and sample outlet, and opening of the air point 2 and sample inlet.

54. A method for harvesting of bulk fluid using a tubing manifold wherein the tubing manifold comprises an air point 1, a sample inlet fluidly coupled to the cell culture vessel, an air point 2, and a sample outlet fluidly coupled to the collection point, each of which is fluidly coupled to the tubing manifold the method comprising:
pumping the bulk volume from the cell culture vessel into the tubing manifold through the sample inlet and concurrently driving the bulk volume out of the tubing manifold through the sample outlet, to the collection point.

55. The method of 54 wherein pumping the bulk volume from the cell culture vessel into the tubing manifold through the sample inlet and concurrently driving the bulk volume out of the tubing manifold through the sample outlet, to the collection point, comprises of closing the air point 1 and air point 2, and opening of the sample inlet and sample outlet.

56. A tubing manifold comprising an air point 1 tube, a sample inlet tube fluidly connected to the cell culture vessel, and air point 2 tube, and a sample outlet tube fluidly connected to the collection point, and a pump section, wherein the tubing manifold is configured to engage the FCS such that each tube engages a respective valve of the FCS and the pump section engages a pump of the FCS.

57. A tubing manifold comprising an air point 1 tube, a sample inlet tube fluidly connected to a sterile connector, and air point 2 tube, and a sample outlet tube fluidly connected to a sterile connector, and a pump section, wherein the tubing manifold is configured to engage the FCS such that each tube engages a respective valve of the FCS and the pump section engages a pump of the FCS.

58. A device for aseptic small volume sampling of liquid, comprising:
a flow control system comprising a pump; and
a tubing manifold,
wherein the tubing manifold comprises a first air point, a sample point, a second air point, and a sample outlet, each of which is fluidly coupled to the tubing manifold, and operatively coupled to the flow control system,
wherein the flow control system (FCS) is operable in a forward flow configuration to pump fluid from the sample point towards the sample outlet,
wherein the flow control system is operable, after pumping the predetermined volume of fluid, to pump air from the first air point towards the sample outlet to drive a predetermined volume of the fluid out of the manifold through the sample outlet, and to prevent additional fluid from the sample point from being pumped towards the sample outlet;
wherein the flow control system is operable, after pumping the predetermined volume of fluid out of the manifold, in a reverse flow configuration to return fluid remaining in the tubing manifold back to the sample point, and
wherein said forward flow and reverse flow configurations substantially prevent air bubble formation in fluid exiting the sample outlet or the sample point.

59. The device of 58, wherein the fluid is a sample bubble of a programmable volume.

60. The device of 59, wherein the programmable volume is consistent through repeated sampling process.

61. The device of 58 or 59, wherein the programmable volume is ranging from 20 μl to 1 ml.

62. The device of 58, wherein the FCS comprises a valve at each of the first air point, sample point, second air point and sample outlet, and a pump, the FCS being configured to control the valves and actuate the pump to draw the fluid into the tubing manifold through the sample point and concurrently drive the air out of the tubing manifold through the second air point.

63. The device of 62, wherein the FCS closes the valve at each of the first air point and the sample outlet, and actuates the pump to rotate in a first direction when drawing the fluid into the tubing manifold through the sample point and concurrently driving the air out of the tubing manifold through the second air point.

64. The device of 63, the FCS being configured to control the valves and actuate the pump to draw fluid into the tubing manifold through the sample point and to drive the predetermined volume of fluid towards the sample outlet.

65. The device of 64, wherein the FCS closes the valve at each of the first air point and the second air point and actuates the pump to rotate in the first direction when drawing fluid into the tubing manifold through the sample point and to drive the predetermined volume of fluid towards the sample outlet.

66. The device of 65, wherein the FCS is configured to control the valves and actuate the pump to draw the air into the tubing manifold through the first air point and concurrently drive the fluid out of the tubing manifold through the sample outlet.

67. The device of 66, wherein the FCS closes the valve at each of the sample point and the second air point and, actuates the pump to rotate in the first direction when drawing the air into the tubing manifold through the first air point and concurrently driving the predetermined volume of fluid out of the tubing manifold through the sample outlet.

68. The device of 67, wherein the FCS is configured to control the valves and actuate the pump to draw air into the tubing manifold through the second air point and concurrently drive the fluid remaining in the tubing manifold out of the manifold through the sample point.

69. The device of 68, wherein the FCS closes the first air point and the sample outlet and actuates the pump to rotate in a second direction opposite the first direction when drawing the air into the tubing manifold through the second air point and concurrently driving the fluid remaining in the tubing manifold out of the manifold through the sample point.

70. The device of 69, wherein the FCS comprises a first sensor positioned adjacent the sample point and a second sensor positioned adjacent the sample outlet, wherein the first sensor and the second sensor are configured detect presence of fluid at the sample point and sample outlet, respectively.

71. The device of 70, wherein the FCS is configured to control the forward flow configuration and the reverse flow configuration in accordance with measurements from the first and second sensors to prevent the formation of air bubbles at the sample point or sample outlet.

72. The device of 70, wherein the FCS uses the first sensor and a flow rate corresponding to a speed of the pump to determine when the fluid enters the tubing manifold and calculating that a particular volume of fluid that is no less than the predetermined volume has entered the tubing manifold from the sample point.

73. The device of 72, wherein the FCS opens the first air point and the sample outlet, closes the sample point and the second air point, and actuates the pump to rotate in the first direction when the first sensor measures that the particular volume of fluid has entered the tubing manifold.

74. The device of 70, the second sensor being configured to determine when the fluid exits the tubing manifold, the FCS determining when the predetermined volume of fluid has exited the tubing manifold.

75. The device of 74, wherein the FCS closes the first air point and the sample outlet, opens the sample point and the second air point, and actuates the pump to rotate in the second direction when the second sensor measures that the predetermined volume of fluid has exited the tubing manifold.

76. The device of 75, the FCS being configured to determine when there is no fluid in the tubing manifold, based on measurements from the first sensor and the second sensor.

77. The device of 76, the FCS closes the first air point, the second air point, the sample point and the sample outlet, and stops the pump on determining that there is no fluid in the tubing manifold.

78. The device of 58, wherein the sample outlet is connected to a series of sample bags or container manifolds.

79. The device of 78, wherein the reverse flow configuration comprises returning fluid that is untouched by the sample bags or container manifolds or the sample outlet to the sample point.

80. The device of 78 or 79, wherein the reverse flow configuration comprises returning fluid that has been separated from the predetermined volume of fluid to the sample point.

81. The device of 58, wherein the predetermined volume of fluid is separated from bulk cell culture media.

82. The device of 81, wherein the flow control system is configured to seal the bulk cell culture media from exposure to contamination at the sample outlet. This can involve closing the sample inlet valve. This can also involve separating the sample bubble (i.e. the sample to be delivered to the sample outlet) from the bulk media. Subsequent to the sample bubble being delivered out the sample outlet, the sample outlet is closed and sterile gas (e.g. sterile air from the second air port) can be used to drive the bulk media back to the culture vessel.

83. The device of 59, wherein the flow control system pumps the predetermined volume of fluid to the sample outlet once the predetermined volume of fluid forms a sample bubble.

84. The device of 58, wherein the sample outlet is exposed to atmosphere.

85. The device of 58, wherein the sample outlet is flexibly adapted to different measurement systems.

86. The device of 58, wherein the tubing manifold is carried in a disposable housing which is attachable to the FCS.

87. The device of 86, wherein the housing comprises apertures that enable the tubing manifold to be located on the FCS and locked in place, operably coupled to a peristaltic pump and pinch valves of the FCS.

88. The device of 87, wherein the tubing manifold is located between the housing and either the pinch valves or the peristaltic pump.

89. The device of 87, wherein the housing comprises grooves that align with the pinch valves, when assembled to the flow control system, for enabling pinching and unpinching of the tubing manifold.

90. A method for aseptic small volume sampling of liquid by using a tubing manifold, wherein the tubing manifold comprises a first air point, a sample point, a second air point, and a sample outlet, each of which is fluidly coupled to the tubing manifold, the method comprising:
pumping a particular volume of fluid into the tubing manifold through the sample point and concurrently driving air out of the tubing manifold through the second air point,
pumping the air into the tubing manifold through the first air point and concurrently driving a predetermined volume of fluid that is not more than the particular volume out of tubing manifold through the sample outlet, and
pumping the air into the tubing manifold through the air outlet and concurrently driving fluid remaining in the tubing manifold out of the tubing manifold through the sample inlet.

91. The method of 90, wherein pumping the particular volume of fluid into the tubing manifold through the sample point and concurrently driving the air out of the tubing manifold through the second air point comprises closing the first air point and the sample outlet.

92. The method of 90, wherein pumping air into the tubing manifold through the first air point and concurrently driving the predetermined volume of fluid out of tubing manifold through the sample outlet comprises closing the second air point and the sample point.

93. The method of 90, wherein pumping air into the tubing manifold through the second air point and concurrently driving fluid remaining in the tubing manifold out of the tubing manifold through the sample point comprises closing the first air point and the sample outlet.

94. A method for aseptic harvesting of liquid by using a tubing manifold, wherein the tubing manifold comprises a sample point, a air point, a sample outlet and a valve at the air point and sample outlet, each of which is fluidly coupled to the tubing manifold, the method comprising:
Closing the valve at the sample outlet and opening the valve at the air point, pumping a predetermined volume of fluid into the tubing manifold through the sample inlet and concurrently driving air out of the tubing manifold through the air point,
closing the valve at the air point and opening the valve at the sample outlet; and
driving the predetermined volume of fluid out of tubing manifold through the sample outlet.

95. A tubing manifold comprising a first air tube, a sample tube, a second air tube, a sample outlet tube, and a pump section, wherein the tubing manifold is configured to engage a flow control system (FCS) such that each of the first air tube, second air tube, sample tube and sample outlet tube engages a respective valve of the FCS and the pump section engages a pump of the FCS.

1. A device for aseptic small volume sampling of liquid, comprising:
a flow control system (FCS) comprising a pump; and
a tubing manifold,
wherein the tubing manifold comprises a first air point, a sample inlet for fluidly coupling the tubing manifold to a cell culture vessel, a second air point, and a sample outlet for fluidly coupling the tubing manifold to a collection point, the tubing manifold being operatively coupled to the FCS when in use, the FCS comprising a plurality of valves operable to selectively open and close the first air point, second air point, sample inlet and sample outlet,
wherein, in use, the FCS operates the pump and valves in a forward flow configuration to pump bulk fluid from the cell culture vessel into the sample inlet such that a predetermined volume of the bulk fluid is pumped to the sample outlet and out of the tubing manifold to the collection point, and a portion of the bulk fluid remains in the tubing manifold,
wherein, in use, the FCS operates the pump and valves in a reverse flow configuration to return the portion of the bulk fluid back to the cell culture vessel without exposing the portion of the bulk fluid to gas.

2. The device of claim 1, wherein the FCS comprises a valve at each of the first air point, sample inlet, second air point, and sample outlet.

3. The device of claim 2, wherein, in the forward flow configuration, the FCS operates the pump and valves to pump the bulk fluid from the cell culture vessel into the tubing manifold through the sample inlet and gas is concurrently driven out of the tubing manifold through the second air point.

4. The device of claim 2, wherein the FCS opens the valve at the sample inlet and second air point, closes the valve at sample outlet and first air point, and actuates the pump to rotate in a first direction when pumping the bulk fluid from the cell culture vessel into the tubing manifold through the sample inlet.

5. The device of claim 2, wherein the FCS is configured to open the valve at the sample inlet and sample outlet, close the valve at the first air point and second air point, and actuate the pump to rotate in a first direction when drawing the bulk fluid from the cell culture vessel through the tubing manifold, through the sample inlet.

6. The device of claim 4, wherein the FCS opens the valve at the sample inlet and sample outlet, closes the valve at the first air point and second air point, and actuates the pump to rotate in the first direction when driving the predetermined volume of the bulk fluid out of the manifold, through the sample outlet, and to the collection point.

7. The device of claim 6, wherein the FCS is configured to control the valve at the sample inlet to separate the bulk fluid pumped into the manifold into the predetermined volume of fluid and the portion of the bulk fluid, operate the valves and actuate the pump to draw gas into the tubing manifold through the first air point and concurrently drive the predetermined volume of fluid out of the manifold to the collection point through the sample outlet, while keeping the portion of the bulk fluid stationary.

8. The device of claim 7, wherein the FCS opens the valve at the sample inlet and the second air point, closes the valve at the first air point and sample outlet, and actuates the pump to rotate in the second direction when driving the portion of the bulk fluid back to the cell culture vessel.

9. The device of claim 8, wherein, in the reverse flow configuration, the FCS controls the valves and actuates the pump in a second direction, opposite to the first direction, to draw gas into the tubing manifold through the second air point and concurrently drive the portion of the bulk fluid out of the manifold, through the sample inlet, back into the cell culture vessel.

10. The device of claim 1, wherein the FCS comprises a first sensor positioned adjacent the sample inlet and a second sensor positioned adjacent the sample outlet, wherein the first sensor and the second sensor are configured to detect presence of fluid at the sample inlet and sample outlet, respectively.

11. The device of claim 10, wherein the FCS is configured to control the forward and reverse flow configurations in accordance with detection of fluid by the first and second sensors.

12. The device of claim 10, wherein the FCS further comprises a timer and is configured to control the forward and reverse flow configurations in accordance with detection of fluid from the first and second sensors and a signal from the timer.

13. The device of claim 1, wherein the tubing manifold comprises two Y-connectors, a first of the Y-connectors comprising the first air point and sample inlet, and a second of the Y-connectors comprising the second air point and sample outlet, the tubing manifold further comprising a pump section engaged, in use, with the pump such that the pump can drive bulk fluid into and through the tubing manifold.

14. The device of claim 1, further comprising a tubing housing for holding the tubing manifold to the FCS.

15. The device of claim 12, wherein the FCS records the amount of time it took to draw the bulk fluid from the cell culture vessel to the first sensor.

16. A method for aseptic small volume fluid sampling using a tubing manifold, the tubing manifold comprising a first air point, a sample inlet fluidly coupled to a cell culture vessel, a second air point, and a sample outlet fluidly coupled to a collection point, the method comprising:
operating in a forward flow configuration by:
pumping a bulk fluid from the cell culture vessel into the tubing manifold through the sample inlet and concurrently driving air out of the tubing manifold through the second air point,
pumping the bulk fluid through the tubing manifold through the sample inlet, and
concurrently driving air out of the tubing manifold through the sample outlet, and
pumping gas into the tubing manifold through the first air point and concurrently driving a predetermined volume of fluid from the bulk fluid out of tubing manifold through the sample outlet, to the collection point while keeping a portion of the bulk fluid stationary in the tubing manifold, and
operating in a reverse flow configuration by pumping the sterile air into the tubing manifold through the second air point and concurrently driving the bulk fluid out of the tubing manifold through the sample inlet, back to the cell culture vessel.

17. The method of claim 16, further comprising sensing a presence of fluid in the tubing manifold at the sample inlet and sample outlet, and controlling pumping in accordance with the presence of fluid in the tubing manifold at the sample inlet and sample outlet.

18. The method of claim 17, wherein the FCS is configured to control the forward flow configuration and the reverse flow configuration in accordance with the presence of fluid in the tubing manifold at the sample inlet and sample outlet to prevent the formation of air bubbles at the sample point or sample outlet.

19. (canceled)

20. A tubing manifold comprising a first air tube, a sample tube, a second air tube, a sample outlet tube, and a pump section fluidly connecting the tubes, wherein the tubing manifold is configured to engage a flow control system (FCS) such that each of the first air tube, second air tube, sample tube and sample outlet tube engages a respective valve of the FCS and the pump section engages a pump of the FCS.

21-95. (canceled)
